(19) United States
(12) Patent Application Publication
(10) Pub. No.: US 2023/0338190 A1
(43) Pub. Date: Oct. 26, 2023

(54) DRIVING A PHACOEMULSIFIER ACTUATOR

(71) Applicant: JOHNSON & JOHNSON SURGICAL VISION, INC., Irvine, CA (US)

(72) Inventors: Amit Fuchs, Hogla (IL); Philip Alper, D.N. Misgav (IL)

(21) Appl. No.: 18/118,326

(22) Filed: Mar. 7, 2023

Related U.S. Application Data
(60) Provisional application No. 63/334,840, filed on Apr. 26, 2022.

(51) Int. Cl. A61F 9/007; B06B 1/02; A61B 17/00

(52) U.S. Cl. CPC A61F 9/00745; B06B 1/0261; B06B 1/0215; A61B 17/00; A61B 2017/00402; A61B 2017/00141; A61B 2017/00194; B06B 2201/55; B06B 2201/76; B06B 2201/40

(57) ABSTRACT

Methods and apparatuses provide a phacoemulsification probe, wherein the probe has a piezoelectric actuator coupled with a needle configured to be inserted into an eye of a patient; and a processor configured to sequentially drive the actuator electrically in a range of frequencies, to measure a respective electrical power input to the actuator at each of the frequencies in the range, to identify a frequency in the range of frequencies wherein a metric of the electrical power input is a maximum, and to estimate from the identified frequency a mechanical resonant frequency of the actuator, and to drive the actuator electrically at the mechanical resonant frequency.

### CROSS-REFERENCE TO RELATED APPLICATIONS

This application claims the benefit of U.S. Provisional Patent Application 63/334,840, filed Apr. 26, 2022, whose disclosure is incorporated herein by reference.

### FIELD OF THE DISCLOSURE

The present disclosure relates generally to eye surgery, and specifically to driving the actuator of a phacoemulsification device used during the surgery.

### BACKGROUND

A cataract is a cloudy area in the lens of an eye that leads to a decrease in vision. Phacoemulsification is a procedure that may be employed to remove an eye lens having a cataract, and a phacoemulsification procedure is typically performed by a surgeon using a handpiece having a piezoelectric actuator. Vibrations from the actuator are transferred into the eye lens and are used to break up and emulsify the lens. The emulsified particles may be removed, and may typically then be replaced by an intra-ocular lens.

### BRIEF DESCRIPTION OF THE DRAWINGS

FIG. 1 is a pictorial view of a phacoemulsification apparatus constructed to operate in accordance with an example of the present disclosure;

FIG. 2A illustrates schematic graphs of electrical parameters vs. frequency for a probe of the apparatus of FIG. 1 with no fluid present;

FIG. 2B illustrates schematic graphs of electrical parameters vs. frequency for the probe with fluid present;

FIG. 3 is a flowchart showing steps of an algorithm performed by a processor of the apparatus of FIG. 1;

FIG. 4 shows a graph of driving frequency vs. time for a probe having different mechanical loads; and

FIG. 5 is a flowchart showing steps of an alternative algorithm performed by the processor.

### DESCRIPTION OF EXAMPLE EMBODIMENTS

### Overview

In a phacoemulsification procedure a needle is inserted into the eye lens of a patient, and the needle is vibrated to break up and emulsify the lens. The emulsified lens particles are then removed from the patient's lens capsule, so that a replacement lens may be inserted into the capsule. In order to generate the vibrations of the needle, the needle is coupled to a piezoelectric actuator, which is driven by an electrically oscillating signal. The electrical signals in turn cause the actuator and its coupled needle to vibrate mechanically.

For the mechanical vibrations to cause the emulsification to be as efficient as possible, the amplitude of the vibrations should be as large as possible, i.e., the combination of the actuator and its coupled needle should be operated at its mechanical resonant frequency. It will be understood that the mechanical resonant frequency of the combination is different from its electrical resonant frequency, and in some cases, such as when the needle contacts the eye lens, the difference is relatively substantial. The mechanical resonant frequency is dependent on mechanical parameters of the combination such as spring constants and the mass contacting the needle; the electrical resonant frequency is dependent on electrical parameters of the actuator such as its inductance, capacitance, and impedance.

The inventors have found that the mechanical resonant frequency may be determined by finding the frequency at which a metric of the electrical power input to the actuator is a maximum. In an example in the present disclosure, the metric comprises the value of the electrical power delivered to the actuator (in terms of the voltage, current and phase) adjusted by a phase factor. Once the mechanical resonant frequency is determined, the actuator may be driven electrically at this frequency.

In order to find the mechanical resonant frequency, in an example in the disclosure, the actuator is electrically driven in a range of frequencies. At each frequency the electrical power input to the actuator is measured, and the frequency wherein the metric of the electrical power input is a maximum is identified. The identified frequency is used to estimate the mechanical resonant frequency of the actuator, and the actuator is driven electrically at the mechanical resonant frequency.

In an alternative example in the disclosure, the actuator is activated electrically, and then the activation is halted. A processor acquires electrical signals generated by the actuator after the activation has been halted, and analyzes the acquired signals so as to identify a mechanical resonant frequency of the actuator. The actuator is then driven at the mechanical resonant frequency.

### DETAILED DESCRIPTION

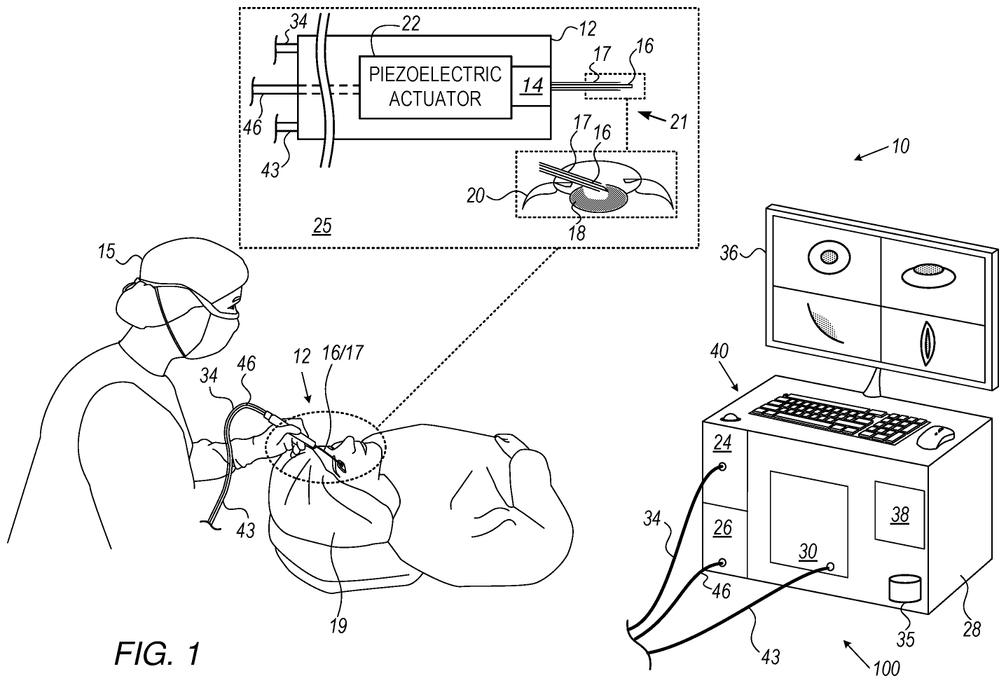

FIG. 1 is a pictorial view of a phacoemulsification apparatus 10 constructed to operate in accordance with an example of the present disclosure. FIG. 1 includes an inset 25, and as shown in the figure and the inset apparatus 10 includes a phacoemulsification probe/handpiece 12 comprising a needle 16 and a coaxial irrigation sleeve 17 that at least partially surrounds the needle 16 and that creates a fluid pathway between the external wall of the needle 16 and the internal wall of the irrigation sleeve 17. Needle 16 is configured to be inserted into a lens capsule 18 of an eye 20 of a patient 19. Needle 16 is mounted on a horn 14 of probe 12 and is shown in inset 25 as a straight needle. However, any suitable needle may be used with the phacoemulsification probe 12, for example, a curved or bent tip needle commercially available from Johnson & Johnson Surgical Vision, Inc, Santa Ana, CA, USA. A physician 15 holds handpiece 12 so as to perform a phacoemulsification procedure on the eye of patient 19. The physician may activate the handpiece using a foot pedal. The foot pedal is not illustrated in FIG. 1.

Handpiece 12 comprises a piezoelectric actuator 22, which is configured to vibrate horn 14 and needle 16 in one or more vibration modes of the combined horn and needle. In the following description, the combined elements are also referred to as vibration elements 21, or just as elements 21. Actuator 22 comprises one or more piezoelectric crystals, which, as is explained in more detail below, are configured to receive one or more signals on respective channels in order to generate desired vibration modes in elements 21. Except where otherwise stated, in the following description the actuator is assumed to receive signals on one channel, so as to generate a linear vibration mode. During the phacoemulsification procedure the vibration of needle 16 is used to break a cataract into small pieces.

During the phacoemulsification procedure, an irrigation sub-system 24, which may be located in a console 28, pumps irrigation fluid from an irrigation reservoir to irrigation sleeve 17 so as to irrigate the eye. The fluid is pumped via an irrigation tubing line 34 running from the console 28 to the probe 12.

An aspiration sub-system 26, also typically located in console 28, aspirates eye fluid and waste matter (e.g., emulsified parts of the cataract) from the patient's eye via needle 16 to a collection receptacle (not shown). Aspiration sub-system 26 comprises a pump which produces a vacuum that is connected from the sub-system to probe 12 by a vacuum aspiration tubing line 46.

Irrigation sub-system 24 and aspiration sub-system 26 are both under control of a processor 38. The processor controls the rate at which the irrigation sub-system pumps its fluid. The processor also controls the vacuum pressure produced by the aspiration sub-system.

Some or all of the functions of processor 38 may be combined in a single physical component or, alternatively, implemented using multiple physical components. The physical components may comprise hard-wired or programmable devices, or a combination of the two. In some examples, at least some of the functions of processor 38 may be carried out by suitable software stored in a memory 35. The software may be downloaded to a device in electronic form, over a network, for example. Alternatively, or additionally, the software may be stored in tangible, non-transitory computer-readable storage media, such as optical, magnetic, or electronic memory.

Processor 38 may receive user-based commands via a user interface 40, which may include setting and/or adjusting a vibration mode and/or a frequency of piezoelectric actuator 22, setting and/or adjusting a stroke amplitude of needle 16, and setting and/or adjusting an irrigation rate and an aspiration rate of irrigation sub-system 24 and aspiration sub-system 26. Additionally, or alternatively, processor 38 may receive user-based commands from controls located in handpiece 12, to, for example, select a trajectory for needle 16.

Processor 38 may present results of the phacoemulsification procedure on a display 36. In an example, user interface 40 and display 36 may be one and the same, such as a touch screen graphical user interface.

The procedure illustrated in FIG. 1 may include further elements, which are omitted for clarity of presentation. For example, physician 15 typically performs the procedure using a stereo-microscope or magnifying glasses, neither of which are shown. Physician 15 may use other surgical tools, in addition to probe 12, which are also not shown to maintain clarity and simplicity.

Console 28 comprises a piezoelectric drive module 30, which is coupled to piezoelectric actuator 22 using electrical wiring running in a cable 43. Drive module 30 is controlled by processor 38 and conveys respective processor-controlled driving signals to each of the receiving channels of actuator 22 via cable 43 to, typically, maintain a distal tip of needle 16 at a maximum vibration amplitude. The drive module may be implemented in hardware or software, for example, in a proportional-integral-derivative (PID) control architecture.

In addition to providing driving signals to actuator 22, module 30, together with processor 38, is able to measure electrical parameters of the signals supplied to the actuator, i.e., for each channel a frequency f, a current I, a voltage V, and a phase α between the voltage and the current are measured.

In order for needle 16 to vibrate at its maximum amplitude, processor 38 operates module 30 to vibrate the needle in mechanical resonance, i.e., the frequency supplied by the module to actuator 22 corresponds to the mechanical resonant frequency of the actuator and its mechanically coupled elements. Such elements include horn 14, needle 16 and irrigation sleeve 17. During a phacoemulsification procedure the mechanically coupled elements may also include irrigation fluid flowing in sleeve 17, aspirated material in needle 16, and material in lens capsule 18 contacted by the irrigation sleeve and/or the needle.

It will be understood that the mechanical resonant frequency of actuator 22 is typically different from the electrical resonant frequency of the actuator. The electrical resonant frequency occurs when the electrical impedance of the actuator is a minimum, causing the amplitude of the electrical signal to be a maximum; the mechanical resonant frequency occurs when the resistance to motion of the actuator and its coupled elements is a minimum, causing the displacement of the actuator needle to be a maximum.

To investigate and clarify the differences between the two resonances, the inventors used probe 12 in a laboratory setup. In the setup, the inventors activated actuator 22 in a single channel, i.e., by using a single pair of electrodes attached to the actuator (typically there are three pairs of electrodes), so that needle 16 vibrated linearly along the longitudinal axis of the needle. The inventors used a stroboscope to measure the displacement of the distal tip of the actuator needle while also measuring electrical parameters provided to the actuator. Using the stroboscope, the inventors were able to determine when the actuator was in mechanical resonance. The inventors found that, as stated above, the mechanical resonant frequency occurs at a different frequency from the electrical resonant frequency. Furthermore, the inventors found that the frequency where an electrical power metric Pₘ, formulated by the inventors, is maximum corresponds to the frequency of maximal distal tip displacement, i.e., to the mechanical resonant frequency.

The electrical power metric Pₘ is equal to a measured power P supplied to the actuator, adjusted by a phase factor ∝ₘ, and is defined by equations (1) and (2):

P=V·I·cos ∝ (1)

Pₘ=V·I·cos(∝+∝ₘ) (2)

where V and I are respectively a root-mean-square voltage and a root-mean-square current supplied to the actuator by module 30, and a is the phase difference, measured by the module, between V and I;

am is a phase adjustment factor that corrects the measured power P so that Pₘ is a maximum when the actuator is operating at its mechanical resonant frequency.

The inventors have found that the value of phase factor ∝ₘ is the same for all channels, and is only dependent on the hardware of the system, i.e., the type of handpiece and actuator 22, since these introduce different delays in measuring the voltage V and current I supplied to the actuator. It will be understood that the value of phase factor ∝ₘ for any handpiece may be determined using the laboratory setup referred to above.

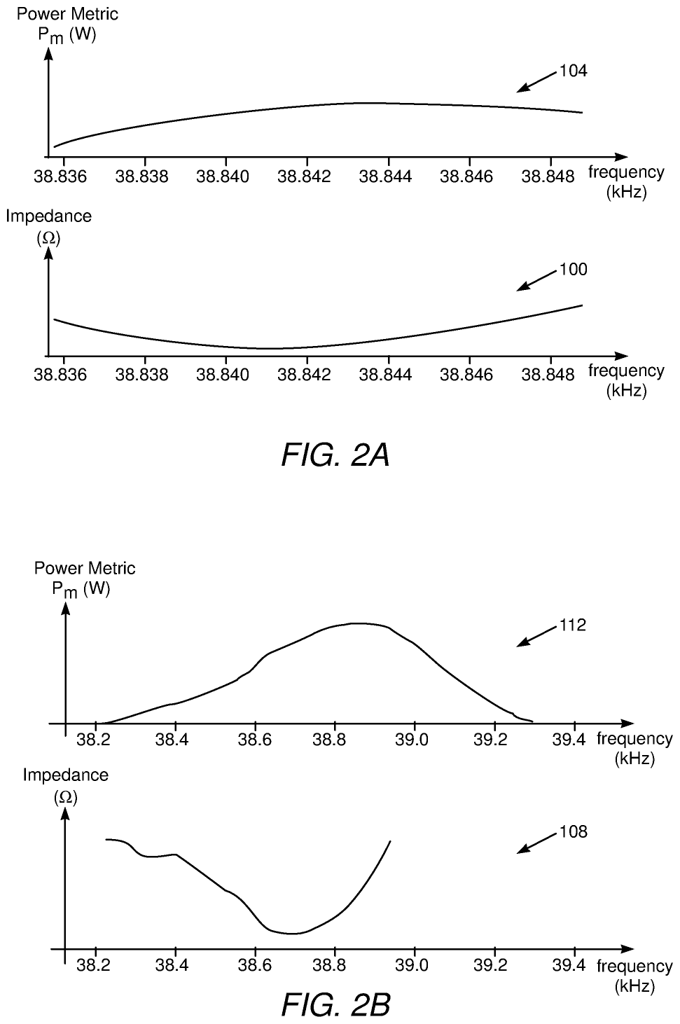

FIG. 2A illustrates schematic graphs of electrical impedance vs. frequency and power metric Pₘ vs. frequency for probe 12 with no fluid in needle 16 and sleeve 17. i.e., when the probe is operated in air. FIG. 2B illustrates schematic graphs of electrical impedance vs. frequency and power metric Pₘ vs. frequency for the probe with fluid present in the needle and the sleeve. In this case there is fluid present in the probe, so that the system is mechanically loaded, but the irrigation and the aspiration lines are not activated. The graphs are generated from data acquired in the lab setup referred to above. Table I lists the resonant frequencies determined for both cases.

TABLE I

| | Electrical Resonant | Mechanical Resonant |
|---|---|---|
| Probe State | Frequency (kHz) | Frequency (kHz) |
| No Fluid | 38.8410 | 38.8438 |
| Fluid | 38.708 | 38.866 |

As is illustrated in FIG. 2A, in a graph 100 the impedance is a minimum at approximately 38.8410 kHz, corresponding to the electrical resonant frequency of the probe with no fluid, and in a graph 104 the power metric Pₘ is a maximum at approximately 38.8438 kHz, corresponding to the probe's mechanical resonant frequency. As is illustrated in FIG. 2B, in a graph 108 the impedance is a minimum at approximately 38.708 kHz, corresponding to the electrical resonant frequency of the probe with fluid, and in a graph 112 the power metric Pₘ is a maximum at approximately 38.866 kHz, corresponding to the mechanical resonant frequency of the probe.

Table I and the graphs of FIGS. 2A and 2B illustrate that the electrical and mechanical resonant frequencies of probe 12 are different, in both the cases of fluid in the probe and no fluid in the probe.

As is described in more detail below, during the phacoemulsification procedure referred to above, module 30 and processor 38 maximize power metric Pₘ to ensure that the actuator is in mechanical resonance.

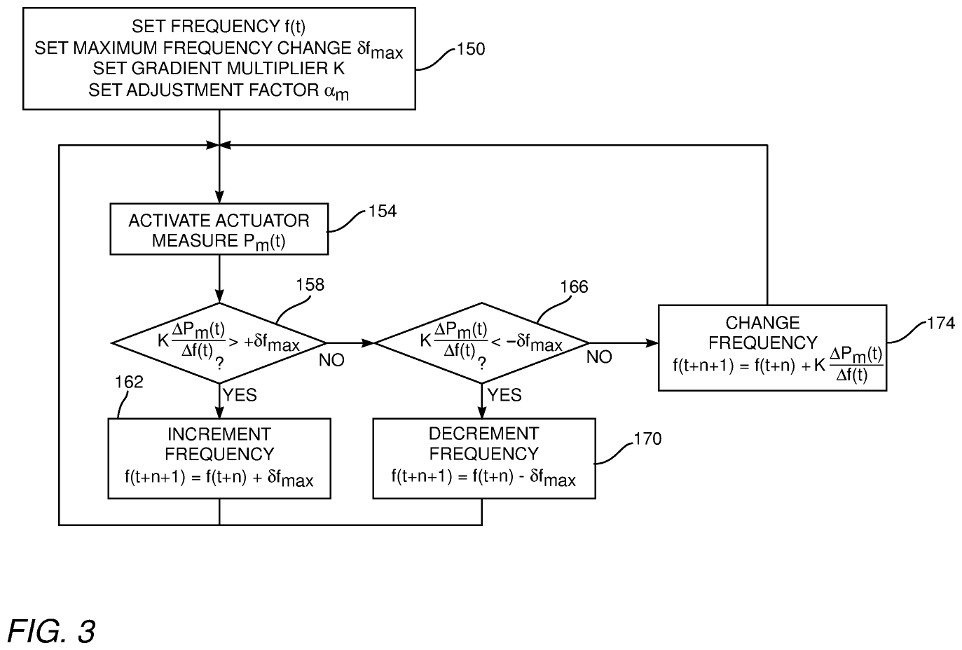

FIG. 3 is a flowchart showing steps of an algorithm performed by processor 38, using module 30, to maximize power metric Pₘ for probe 12 during a phacoemulsification procedure. The flowchart is drawn assuming that a single channel of actuator 22 of the probe is energized. The maximization is performed by sequentially altering a frequency f(t) applied to the probe, where f(t) is the frequency of the signal applied to the actuator of the probe at a time t, and Pₘ(t) is the power metric at time t. The algorithm finds the frequency f(t) at which the gradient

$$\frac{Δ {P}_{m} ( t )}{Δ f ( t )}$$

is zero, and in some examples when the graph of the power metric Pₘ vs. frequency f is concave down, and this frequency is the mechanical resonant frequency of the probe. Processor 38 and module 30 drive actuator 22 at the mechanical resonant frequency so that the actuator is in mechanical resonance. (Applying the concave down condition ensures that the algorithm converges faster.)

In an initial step 150, at a time t=0, an operator of the algorithm, or physician 15, inputs to processor 38 an initial value of the frequency f(0), assumed to be close to an actual value of the mechanical resonant frequency of the actuator of probe 12. In one example f(0) is set at 38 kHz, but the initial value may be larger or smaller than this.

Alternatively, the initial value of f(0) may be determined by implementing a fast wide frequency sweep to find an approximate frequency giving a minimum impedance for the probe. Such a sweep is described in U.S. application Ser. No. 17/529,713, which is assigned to the assignee of the present disclosure.

Further alternatively, the initial value of f(0) may be preprogrammed from a calibration of the type of probe 12. In this case the value may be stored in memory 35.

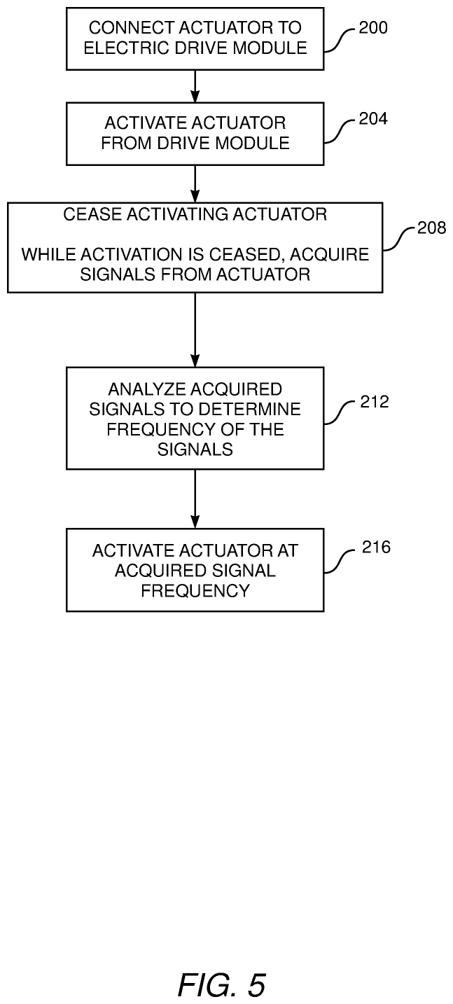

As a yet further example, the initial value of f(0) may be found using the alternative algorithm described below with reference to FIG. 5.

A value for a maximum frequency change δfₘₐₓ that the processor is able to make during an iterative phase of the algorithm is input to the processor. In one example δfₘₐₓ=2 10 Hz; in another example δfₘₐₓ=10 Hz. However, δfₘₐₓ may be larger or smaller than these values. Typically, for a larger value of δfₘₐₓ the system converges faster but is more sensitive to noise, whereas for a smaller value of δfₘₐₓ the system converges slower and is less sensitive to noise.

An additional input to the processor is a value for a gradient multiplier K. As is described hereinbelow, the processor iteratively calculates the gradient

$$\frac{Δ {P}_{m} ( t )}{Δ f ( t )} ,$$

and the processor multiplies the calculated gradient by K to determine a frequency that is compared with δfₘₐₓ In one example K is 0.02 Hz².W⁻¹. However, K may be larger or smaller than this value.

As for δfₘₐₓ a larger value of K enables the system to converge faster while being more sensitive to noise, while a smaller value of K enables slower convergence but less sensitivity for noise. One having ordinary skill in the art will be able to select optimal values for δfₘₐₓ and K without undue experimentation.

In addition, a value of adjustment factor αₘ, as determined above, is input to the processor. In one example, αₘ is in a range from approximately 2° to approximately 15°, but in other examples αₘ may be outside this range.

The processor 38 continues to a measurement step 154, where the processor 38 and module 30, on receipt of a control signal from physician 15 (e.g., actuation of a foot pedal), begin to drive actuator 22 at the frequency f(0) set in step 150. Herein the actuator is assumed to be driven in a single channel so that needle 16 vibrates linearly, and the case where the actuator is driven by multiple channels is addressed separately below. Once actuator 22 is driven, the processor 38 and module 30 measure the values of V, I, and a, as defined above for equations (1) and (2), and use these values to calculate a value of power metric Pₘ(t).

In a first decision step 158, the processor calculates a value of

$$K \frac{Δ {P}_{m} ( t )}{Δ f ( t )}$$

and compares the calculated value to +5δfₘₐₓ, by analyzing an expression

$$K \frac{Δ {P}_{m} ( t )}{Δ f ( t )} > + δ {f}_{max} \qquad ( 3 )$$

(When the algorithm initiates,

$$K \frac{Δ {P}_{m} ( t )}{Δ f ( t )}$$

is not defined, so initially in step 158 the processor is configured to automatically set its value to be larger than δfₘₐₓ.) If decision step 158 returns positive, i.e., yes, processor 38 proceeds to a frequency increment step 162 wherein the processor and module increase the frequency driving the actuator by δfₘₐₓ It will be understood that a positive return for decision step 158 indicates that the gradient

$$\frac{Δ {P}_{m} ( t )}{Δ f ( t )}$$

is positive. If decision step 158 returns negative, i.e., no, processor 38 continues to a second decision step 166.

In second decision step 166 the processor 38 compares the calculated value of

$$K \frac{Δ {P}_{m} ( t )}{Δ f ( t )} to - δ {f}_{max} ,$$

by analyzing an expression

$$K \frac{Δ {P}_{m} ( t )}{Δ f ( t )} < - δ {f}_{max} \qquad ( 4 )$$

If decision step 166 returns positive, i.e., yes, processor 38 proceeds to a frequency decrement step 170 wherein the processor 38 and module 30 decrease the frequency driving the actuator by δfₘₐₓ It will be understood that a positive return for decision step 166 indicates that the gradient

$$\frac{Δ {P}_{m} ( t )}{Δ f ( t )}$$

is negative.

If second decision step 166 returns negative, i.e., no, processor 38 continues to a change frequency step 174, wherein the processor changes the frequency driving the actuator by the value

$$K \frac{Δ {P}_{m} ( t )}{Δ f ( t )} .$$

Since step 174 follows from both decision steps 158 and 166 returning negative, expression (5) applies:

$$- δ {f}_{max} \leq K \frac{Δ {P}_{m} ( t )}{Δ f ( t )} \leq + δ {f}_{max} \qquad ( 5 )$$

I.e., the frequency change applied to the actuator in step 174 is an increment or a decrement, but no more than |δfₘₐₓ|.

From steps 162, 170, and 174 processor 38 returns to step 154, so that these steps, together with decisions 158 and 166, form an iterative process. The frequency changes in steps 162, 170, and 174 are given respectively by equations (6), (7), and (8), where n is a counter of the iteration.

$$Step 162 : f ( t + n + 1 ) = f ( t + n ) + δ {f}_{max} \qquad ( 6 )$$

$$Step 170 : f ( t + n + 1 ) = f ( t + n ) - δ {f}_{max} \qquad ( 7 )$$

$$Step 174 : f ( t + n + 1 ) = f ( t + n ) + K \frac{Δ {P}_{m} ( t )}{Δ f ( t )} \qquad ( 8 )$$

The iterative process described above, wherein the frequency changes correspond to regions of the graph of power metric Pₘ vs. frequency f, works optimally when the graph is concave down, i.e., when the frequency changes made by the process are proximate to a maximum value of Pₘ. However, it will be understood that the iterative process operates when the concave down property is not present.

Furthermore, if the mechanical resonant frequency of the probe is constant, the frequency generated by the iterative process, and input to the actuator of the probe, is from step 174 and this frequency substantially corresponds to the mechanical resonant frequency, and typically differs from it by at most ±δfₘₐₓ.

If the mechanical resonant frequency of probe 12 changes by a relatively large amount, for example because of a change in a rate of flow of fluid in aspiration line 46 and/or irrigation line 34, and/or because material contacting needle 16 changes, the increment or decrement of decisions 158 and 166 mean that the iterative process quickly adjusts to the new frequency. Decision steps 158 and 166 also act to improve the system's toleration of noise.

If the mechanical resonant frequency changes by a relatively small amount, typically only change frequency step 174 of the iterative process is invoked, the process is also quick.

Thus, for both small and large changes of the probe mechanical resonant frequency, the iterative process of the algorithm quickly determines the new resonant frequency used to drive the probe.

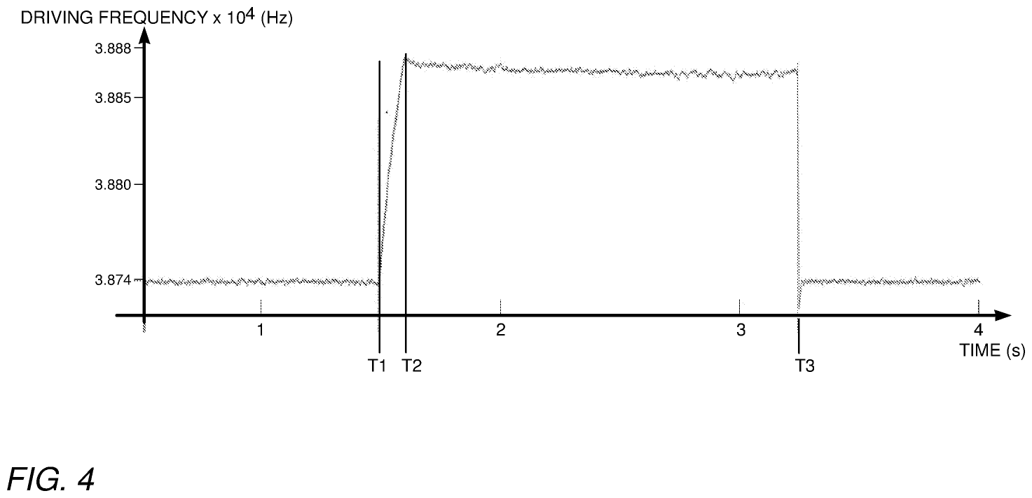

FIG. 4 shows a graph of driving frequency vs. time for a probe 12 having different mechanical loads. Up until a time T1 the probe has no load applied and using the flowchart of FIG. 3 processor 38 operates at a resonant frequency of approximately 3.874×10⁴ Hz. At the time T1 a load is applied to the probe and the processor changes the frequency, using the flowchart, and continues to change the frequency until a new resonant frequency is found. This occurs at a time T2, showing the new resonant frequency is approximately 3.887×10⁴ Hz. At a time T3 the load is removed, so the processor returns to operate the resonant frequency at approximately 3.874×10⁴ Hz.

The graph illustrates that the times to change resonant frequencies are small: the time from T1 to T2 is of the order of 0.1 s, the time for changing at T3 is of the order of 0.01 s.

The description above assumes that actuator 22 is energized in one channel. If the actuator 22 is energized in more than one channel, then the processor 38 and module 30 calculate a value for the power metric Pₘ for each of the channels, and in one example the processor sums the power metrics and finds the frequency where the sum is a maximum. This frequency is the mechanical resonant frequency of the probe.

Thus, when the actuator is activated in c channels, expressions (3), (4), (5) and (8) respectively become:

$$K \frac{Δ {\sum }_{c} {P}_{m} ( t )}{Δ f ( t )} > + δ {f}_{max} \qquad ( {3}^{′} )$$

$$K \frac{Δ {\sum }_{c} {P}_{m} ( t )}{Δ f ( t )} < - δ {f}_{max} \qquad ( {4}^{′} )$$

$$- δ {f}_{max} \leq K \frac{Δ {\sum }_{c} {P}_{m} ( t )}{Δ f ( t )} \leq + δ {f}_{max} \qquad ( {5}^{′} )$$

$$Step 174 : f ( t + n + 1 ) = f ( t + n ) + K \frac{Δ {\sum }_{c} {P}_{m} ( t )}{Δ f ( t )} \qquad ( {8}^{′} )$$

Typically, K is the same for all channels. ° m for each channel may be determined as described above for the case of a single channel.

FIG. 5 is a flowchart showing steps of an alternative algorithm performed by processor 38, using module 30, to determine the mechanical resonant frequency of probe 12, and to drive the probe at this frequency so that it is in resonance. As is described hereinbelow, in the alternative algorithm, probe 12 is induced to vibrate mechanically. The frequency of the induced vibration, which is the mechanical resonant frequency, is measured, and the processor activates the probe at this frequency, so the probe is in mechanical resonance.

In an initial step 200 of the flowchart, probe 12 is coupled to console 28, so that actuator 22 is connected to processor 38 and drive module 30, as is described above with reference to FIG. 1.

In an electrical activation step 204, the processor and the drive module activate actuator 22 electrically, in response to a command from physician 15. The electrical signal for the activation may comprise a sinusoidal signal, in one example at a frequency selected by the physician, or alternatively at a preset frequency, so that the actuator vibrates mechanically at the selected frequency. Alternatively, the electrical signal may comprise a pulse which compresses and/or extends the piezoelectric elements of the actuator.

In a cease activation step 208, processor 38 terminates the electrical activation of step 204 of the actuator. On termination, the processor begins acquiring and storing signals from the actuator. It will be understood that at termination of the electrical activation the actuator is not in a mechanical equilibrium state, so that at the termination the actuator begins to return to an equilibrium state, i.e., a state where the piezoelectric elements are neither compressed nor extended. The process of returning to the equilibrium state causes the actuator to vibrate at the mechanical resonant frequency of the actuator, and the vibrations cause the actuator piezoelectric elements to generate the signals that the processor acquires.

In an analysis step 212 the processor analyzes the signals stored in step 208, to ascertain the frequency of the signals, i.e., the mechanical resonant frequency.

In an activation step 216, the processor activates the actuator at the acquired signal frequency, i.e., at the mechanical resonant frequency of the actuator.

### EXAMPLES

### Example 1

An apparatus, comprising: a phacoemulsification probe (12), comprising a piezoelectric actuator (22) coupled with a needle (16) configured to be inserted into an eye (20) of a patient (19); and a processor (38) configured: to sequentially drive the actuator (22) electrically in a range of frequencies, to measure a respective electrical power input to the actuator (22) at each of the frequencies in the range, to identify a frequency in the range of frequencies wherein a metric of the electrical power input is a maximum, and to estimate from the identified frequency a mechanical resonant frequency of the actuator, and to drive the actuator (22) electrically at the mechanical resonant frequency.

### Example 2

The apparatus according to Example 1, wherein sequentially driving the actuator (22) in the range of frequencies comprises inputting respective signals to the actuator (22) at each of the frequencies, and wherein the processor (38) is configured to calculate the measured electrical power input of a given signal as a product V·I·cos ∝ wherein V is a voltage, I is a current, and a is a phase between the voltage and the current of the given signal.

### Example 3

The apparatus of Example 2, wherein the metric is a product V·I·cos(∝+∝ₘ) wherein αm is a phase adjustment factor that corrects the measured electrical power input so that the metric is a maximum when the actuator is operating at the mechanical resonant frequency.

### Example 4

The apparatus of any of Example 1 or Example 2, wherein identifying the frequency comprises measuring a gradient comprising a change of the metric divided by a change of the frequency, and determining the frequency at which the gradient is zero.

### Example 5

The apparatus of Example 4, wherein measuring the gradient comprises iteratively measuring the gradient while sequentially driving the actuator at each of the frequencies in the range of frequencies.

### Example 6

The apparatus of any of Example 1 to Example 5, wherein the actuator (22) is configured to be energized in a single channel.

### Example 7

The apparatus of any of Example 1 to Example 6, wherein the actuator (22) is configured to be energized in a plurality of channels, and wherein the processor is configured to identify the frequency wherein a sum of the metrics of the electrical power input for each channel is a maximum.

### Example 8

A method, comprising: coupling a piezoelectric actuator (22), comprised in a phacoemulsification probe (12), with a needle (16) configured to be inserted into an eye (20) of a patient (19); sequentially driving the actuator (22) electrically in a range of frequencies; measuring a respective electrical power input to the actuator (22) at each of the frequencies in the range; identifying a frequency in the range of frequencies wherein a metric of the electrical power input is a maximum; estimating from the identified frequency a mechanical resonant frequency of the actuator (22); and driving the actuator (22) electrically at the mechanical resonant frequency.

### Example 9

The method of Example 8, wherein sequentially driving the actuator (22) in the range of frequencies comprises inputting respective signals to the actuator at each of the frequencies, and measuring the respective electrical power comprises calculating the measured electrical power input of a given signal as a product V·I·cos ∝ wherein V is a voltage, I is a current, and a is a phase between the voltage and the current of the given signal.

### Example 10

The method of Example 9, wherein the metric is a product V·I·cos(∝+∝ₘ) wherein αm is a phase adjustment factor that corrects the measured electrical power input so that the metric is a maximum when the actuator (22) is operating at the mechanical resonant frequency.

### Example 11

The method of any of Example 8 to Example 10, wherein identifying the frequency comprises measuring a gradient comprising a change of the metric divided by a change of the frequency, and determining the frequency at which the gradient is zero.

### Example 12

The method of Example 11, wherein measuring the gradient comprises iteratively measuring the gradient while sequentially driving the actuator (22) at each of the frequencies in the range of frequencies.

### Example 13

The method of any of Example 8 to Example 12, wherein the actuator (22) is configured to be energized in a single channel.

### Example 14

The method of any of Example 8 to Example 13, wherein the actuator (22) is configured to be energized in a plurality of channels, and wherein identifying the frequency comprises identifying the frequency wherein a sum of the metrics of the electrical power input for each channel is a maximum.

### Example 15

An apparatus, comprising: a phacoemulsification probe (12), comprising a piezoelectric actuator (22) coupled with a needle (16) configured to be inserted into an eye (20) of a patient (19); and a processor (38) configured: to activate the actuator (22) electrically, and subsequently halt activation of the actuator (22), to acquire electrical signals generated by the actuator (22) after halting the activation; to analyze the acquired signals so as identify therefrom a mechanical resonant frequency of the actuator (22), and to drive the actuator (22) electrically at the identified mechanical resonant frequency.

### Example 16

The apparatus of Example 15, wherein the activation comprises activation with an oscillating signal.

### Example 17

The apparatus of Example 15 or Example 16, wherein the activation comprises activation with an electric pulse.

### Example 18

A method, comprising: coupling a piezoelectric actuator (22), comprised in a phacoemulsification probe (12), with a needle (16) configured to be inserted into an eye (20) of a patient (19); activating the actuator (22) electrically, and subsequently halting activation of the actuator (22), acquiring electrical signals generated by the actuator (22) after halting the activation; analyzing the acquired signals so as identify therefrom a mechanical resonant frequency of the actuator (22), and driving the actuator (22) electrically at the identified mechanical resonant frequency.

### Example 19

The method of Example 18, wherein activating the actuator (22) comprises activating with an oscillating signal.

### Example 20

The method of Example 18 or Example 19, wherein activating the actuator (22) comprises activating with an electric pulse.

It will be appreciated that the examples described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. Apparatus, comprising:
a phacoemulsification probe, comprising a piezoelectric actuator coupled with a needle configured to be inserted into an eye of a patient; and
a processor configured:
to sequentially drive the actuator electrically in a range of frequencies,
to measure a respective electrical power input to the actuator at each of the frequencies in the range,
to identify a frequency in the range of frequencies wherein a metric of the electrical power input is a maximum, and to estimate from the identified frequency a mechanical resonant frequency of the actuator, and
to drive the actuator electrically at the mechanical resonant frequency.

2. The apparatus according to claim 1, wherein sequentially driving the actuator in the range of frequencies comprises inputting respective signals to the actuator at each of the frequencies, and wherein the processor is configured to calculate the measured electrical power input of a given signal as a product V·I·cos ∝ wherein V is a voltage, I is a current, and α is a phase between the voltage and the current of the given signal.

3. The apparatus according to claim 2, wherein the metric is a product V·I·cos(∝+∝ₘ) wherein αₘ is a phase adjustment factor that corrects the measured electrical power input so that the metric is a maximum when the actuator is operating at the mechanical resonant frequency

4. The apparatus according to claim 1, wherein identifying the frequency comprises measuring a gradient comprising a change of the metric divided by a change of the frequency, and determining the frequency at which the gradient is zero.

5. The apparatus according to claim 4, wherein measuring the gradient comprises iteratively measuring the gradient while sequentially driving the actuator at each of the frequencies in the range of frequencies.

6. The apparatus according to claim 1, wherein the actuator is configured to be energized in a single channel.

7. The apparatus according to claim 1, wherein the actuator is configured to be energized in a plurality of channels, and wherein the processor is configured to identify the frequency wherein a sum of the metrics of the electrical power input for each channel is a maximum.

8. A method, comprising:
coupling a piezoelectric actuator, comprised in a phacoemulsification probe, with a needle configured to be inserted into an eye of a patient;
sequentially driving the actuator electrically in a range of frequencies;
measuring a respective electrical power input to the actuator at each of the frequencies in the range;
identifying a frequency in the range of frequencies wherein a metric of the electrical power input is a maximum;
estimating from the identified frequency a mechanical resonant frequency of the actuator; and
driving the actuator electrically at the mechanical resonant frequency.

9. The method according to claim 8, wherein sequentially driving the actuator in the range of frequencies comprises inputting respective signals to the actuator at each of the frequencies, and measuring the respective electrical power comprises calculating the measured electrical power input of a given signal as a product V·I·cos ∝ wherein V is a voltage, I is a current, and a is a phase between the voltage and the current of the given signal.

10. The method according to claim 9, wherein the metric is a product V·I·cos(∝+∝ₘ) wherein αₘ is a phase adjustment factor that corrects the measured electrical power input so that the metric is a maximum when the actuator is operating at the mechanical resonant frequency.

11. The method according to claim 8, wherein identifying the frequency comprises measuring a gradient comprising a change of the metric divided by a change of the frequency, and determining the frequency at which the gradient is zero.

12. The method according to claim 11, wherein measuring the gradient comprises iteratively measuring the gradient while sequentially driving the actuator at each of the frequencies in the range of frequencies.

13. The method according to claim 8, wherein the actuator is configured to be energized in a single channel.

14. The method according to claim 8, wherein the actuator is configured to be energized in a plurality of channels, and wherein identifying the frequency comprises identifying the frequency wherein a sum of the metrics of the electrical power input for each channel is a maximum.

15. Apparatus, comprising:
a phacoemulsification probe, comprising a piezoelectric actuator coupled with a needle configured to be inserted into an eye of a patient; and
a processor configured:
to activate the actuator electrically, and subsequently halt activation of the actuator,
to acquire electrical signals generated by the actuator after halting the activation;
to analyze the acquired signals so as identify therefrom a mechanical resonant frequency of the actuator, and
to drive the actuator electrically at the identified mechanical resonant frequency.

16. The apparatus according to claim 15, wherein the activation comprises activation with an oscillating signal.

17. The apparatus according to claim 15, wherein the activation comprises activation with an electric pulse.

18. A method, comprising:
coupling a piezoelectric actuator, comprised in a phacoemulsification probe, with a needle configured to be inserted into an eye of a patient;
activating the actuator electrically, and subsequently halting activation of the actuator,
acquiring electrical signals generated by the actuator after halting the activation;
analyzing the acquired signals so as identify therefrom a mechanical resonant frequency of the actuator, and
driving the actuator electrically at the identified mechanical resonant frequency.

19. The method according to claim 18, wherein activating the actuator comprises activating with an oscillating signal.

20. The method according to claim 18, wherein activating the actuator comprises activating with an electric pulse.
